(19) United States
(12) Patent Application Publication
(10) Pub. No.: US 2025/0322478 A1
(43) Pub. Date: Oct. 16, 2025

(54) SAFETY AND WELLNESS SYSTEM

(71) Applicant: Kokomo Solutions, Inc., Northbrook, IL (US)

(72) Inventor: Daniel Jongdae Lee, Northbrook, IL (US)

(21) Appl. No.: 19/175,953

(22) Filed: Apr. 10, 2025

Related U.S. Application Data
(60) Provisional application No. 63/632,422, filed on Apr. 10, 2024.

(51) Int. Cl. G06Q 50/26; G06Q 50/43; G16H 80/00

(52) U.S. Cl. CPC G06Q 50/265; G06Q 50/43; G16H 80/00

(57) ABSTRACT

A robust and easy-to-use health, safety, and wellness platform that focuses on individual safety and community safety. A cloud-based platform facilitates the health, safety, and wellness of individuals in a user community. The platform comprises at least several features, provided as modules within the platform, including an incident management feature having a centralized dashboard for collecting, tracking, managing, and responding to events, an anonymous reporting feature providing individual users in the user community with a safe and private means for reporting concerning behavior, a digital emergency alert button, a data collection feature including customizable surveys to collect information from community users, a communication management feature to keep community users informed with important update information, and a guest and visitor management feature to ensure all non-community users (guests or visitors) are properly managed through screening and tracking.

### CROSS-REFERENCE TO RELATED APPLICATION

The present disclosure claims the benefit of Provisional Patent Application No. 63/632,422, filed on Apr. 10, 2024, and entitled “SAFETY AND WELLNESS SYSTEM”, the contents of which are incorporated by reference in its entirety.

### TECHNICAL FIELD

The present invention relates to systems and methods for a safety and wellness platform for use in businesses and schools. In embodiments, the invention relates to a cloud-based system for gathering information and connecting individuals in real-time to provide safety and insure well-being.

### BACKGROUND

When it comes to safety and well-being, businesses and schools are unprepared for the emergencies and chaos that comes from a lack of information collection and central connectivity. That is, the information exists and there is a certain amount of reporting that may take place when incidents occur. However, making information gathering easier, and in some cases anonymous, and placing it all in one central location is a critical first step to building a robust and easy-to-use health, safety, and wellness system. It seems many businesses and school systems are unaware of the information available, or how to collect and make sense of the information they have. Furthermore, the consequences of being unprepared can be devastating to a business, school, and community.

### SUMMARY OF THE INVENTION

This disclosure relates to systems and methods for a safety and wellness platform for the centralized management of aspects of a businesses or school.

According to an aspect of the present disclosure, a cloud-based system includes an incident management system having a centralized dashboard for collecting, tracking, managing, and responding to events. The cloud-based system also includes an anonymous reporting system for providing individual users in the user community with a safe and private means for reporting concerning behavior. The cloud-based system further includes an emergency alter system including a mobile application with an emergency alert button for indicating there is an emergency. The cloud-based system also comprises a data collection system including customizable surveys to collect information from community users and a communication management system to keep community users informed. The cloud-based system includes a visitor management system including a guest registration system and a background check for ensuring a visitor is safe through screening and tracking.

According to an aspect of the present disclosure, a method for facilitating health, safety, and wellness of individuals in a user community includes the steps of: managing incidents using a centralized dashboard for collecting, tracking, managing, and responding to events; allowing anonymous reporting by providing individual users in the user community; providing emergency alter system including a digital emergency alert button for indicating there is an emergency; collecting data from community users by customizable surveys; managing communication to keep community users informed with important update information; and managing visitors to ensure all visitors are safe and monitored while at the community through screening and tracking.

According to an aspect of the present disclosure, a cloud-based system for facilitating health, safety, and wellness of individuals in a user community includes an incident management system having a centralized dashboard for collecting, tracking, managing, and responding to events, wherein the incident management system is configured to predict future incidents using artificial intelligence (AI), and wherein the AI is programed to determine a possible or likely next sequence of events based on collected incident events and personal data. The cloud-based system further includes a safety alert system including an anonymous reporting system for allowing users to anonymously report incidents and an emergency alter system for remotely indicating a user is experiencing an emergency. The cloud-based system also includes a safety operation center system for monitoring the community and communicating with law enforcement. The safety operation center system includes: a cyber-incident management system configured to monitor the use of community networks, cyber threats, and cyber bullying; and a ridership management system configured to monitor user usage of community vehicles to ensure that the user safely boards and de-boards from the vehicles.

### BRIEF DESCRIPTION OF THE DRAWINGS

For the purpose of facilitating an understanding of the subject matter sought to be protected, there are illustrated in the accompanying drawings, embodiments thereof, from an inspection of which, when considered in connection with the following description, the subject matter sought to be protected, its construction and operation, and many of its advantages should be readily understood and appreciated.

FIG. 1 illustrates a block diagram of a system including a cloud-based platform for facilitating health, safety, and wellness of individuals in a user community, according to the principles of the present disclosure;

FIG. 2 illustrates the system 100 and a method 2000, according to the principles of the present disclosure;

FIG. 3 illustrates an incident management system including a dashboard, according to the principles of the present disclosure;

FIGS. 4 and 5 illustrate the incident management system configured to implement predictive AI modeling, according to the principles of the present disclosure;

FIGS. 6 and 7 illustrate the incident management system including a patrolling management system 220 is configured to track and manage staff patrols or inspections of sites and facilities, according to the principles of the present disclosure;

FIGS. 8 and 9 illustrate the incident management system including facility and asset management system 230 is configured to manage and monitor all facilities and assets, according to the principles of the present disclosure;

FIGS. 10-12 illustrate a visitor management system including a guest registration 410, according to the principles of the present disclosure;

FIGS. 13-16 illustrate a safety alert system including a reporting system and an emergency alert system, according to the principles of the present disclosure;

FIGS. 17 and 18 illustrate a health and wellness system, according to the principles of the present disclosure;

FIGS. 19 and 20 illustrate a tardy management system, according to the principles of the present disclosure;

FIGS. 21 and 22 illustrate a classroom observation management system, according to the principles of the present disclosure; and

FIGS. 23-26 illustrate a survey system, according to the principles of the present disclosure.

### DETAILED DESCRIPTION OF THE ENABLING EMBODIMENTS

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative bases for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical application. Various combinations and modifications of the features consistent with the teachings of this disclosure, however, could be desired for particular applications or implementations.

The following detailed description is merely exemplary in nature and is not intended to limit the present disclosure or uses thereof. The systems and methods are described herein with respect to schools and student safety. However, one of ordinary skill in the art will understand that the systems and methods may also be applied to and used for any facility, collection of facilities, community, or organization where there is a need to maintain safety, confidentiality, trade secrets, etc. (e.g., facilities including businesses, universities, entertainment venues, hospitals, research facilities, etc.).

Businesses and schools often lack the necessary information collection and centralized communication network for effective an emergency response. While some reporting of incidents occurs, many of those monitoring the information are unaware of its availability or how to use the information, leading to potentially devastating consequences. Therefore, making information gathering easier and more centralized is crucial for a robust health, safety, and wellness system for businesses, schools, and other communities.

With regards to schools in the United States, between 2000 and 2022, 1512 people were killed or wounded by gunfire on school grounds, with 814 of these incidents occurring between 2017 and 2022. In addition, research shows that attackers tell others about their plans in four out of five school shootings and, in nine out of ten violent school incidents, the assailant had displayed objectively concerning or prohibited behaviors that were observed by others. Effectively collecting and analyzing reports and violent incidents can be used to prevent serious harm from occurring. Part of effectively collecting reports of potential attacks includes facilitating an easy method of reporting and establishing a trusted, confidential means by which a user can report their concerns. Certain studies have shown that users are more likely to report threatening behavior if such reporting is anonymous.

There is also a need to manage facilities, assets, and events and provide access to key programs, such as wellness and bus programs for students. With regards to wellness programs, school districts often lack the technical resources to securely manage student immunization requirements or provide students with proper health resources. School districts can also find it difficult to efficient manage all of their facilities, software licenses, repairs, and events, as well as to keep track of students using busing programs so as to ensure student safety. Accordingly, centralizing these important systems into a single platform can result in efficient management, communication, confidentiality, and can also significantly reduce costs to the school district.

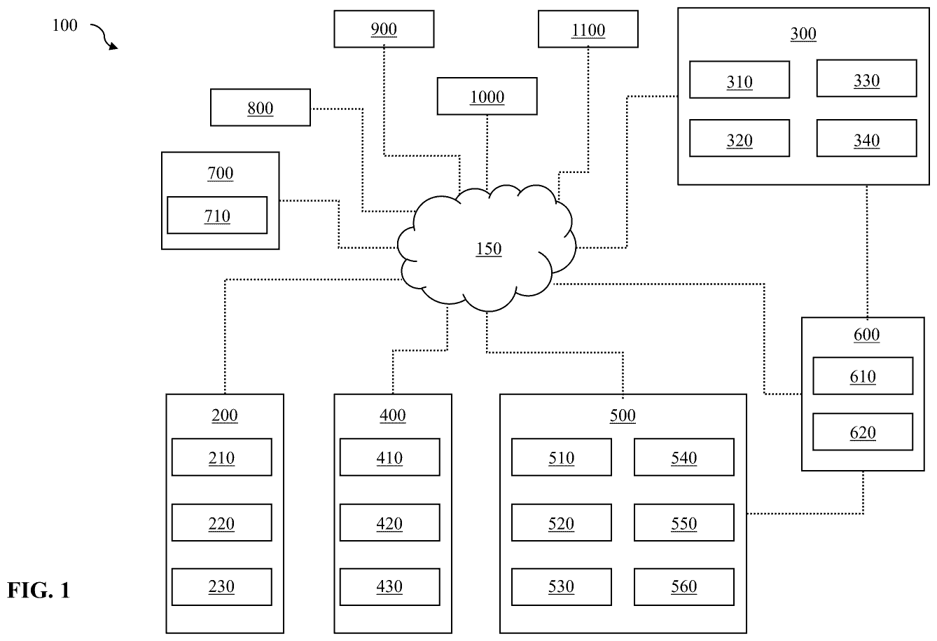

FIG. 1 is a block diagram that generally illustrates a system 100 with a centralized platform for facilitating the health, safety, and wellness of individuals, according to an embodiment of the present disclosure. The system 100 may include one or more databases, one or more application program interfaces (API), a web based front end, and/or a backend platform. The system 100 may comprise various systems, such as computing systems, cloud based systems, Internet based systems, and/or other suitable systems. The databases associated with the system 100 may store encrypted documents, non-encrypted documents, other suitable documents, and/or a combination thereof. Architecture associated with the system 100 is adapted and/or configured to meet all technology requirements of a centralized platform for managing the health, safety, and wellness of individuals in a school, business, community, or the like. The system may include electronic computing and communications system, or variations thereof, or the system 100 may include, a distributed computing system (e.g., a client-server computing system), a cloud computing system, a clustered computing system, or the like.

Users of the system 100 may include administrators, IT personnel, safety officers, counselors, psychologists, teachers, students, parents, or the like. The users may access and use the system 100 via a mobile phone, a tablet computer, a laptop computer, a notebook computer, a desktop computer, or any other suitable computing device or combination of computing devices.

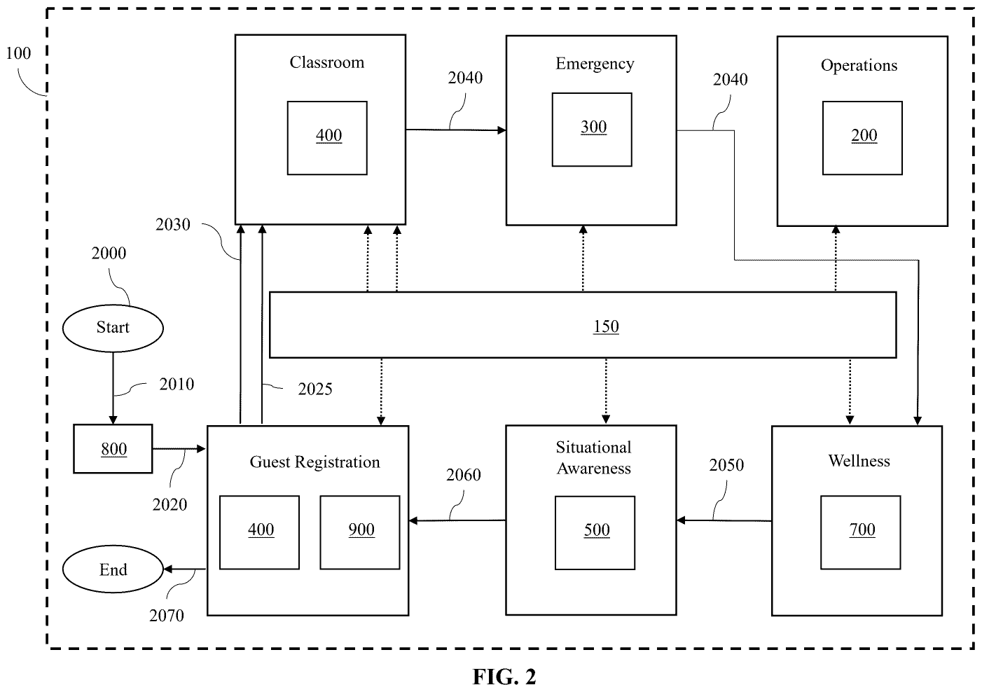

Referring to FIGS. 1 and 2, the system 100 includes a cloud-based platform 150 comprising various modules for facilitating the health, safety, and wellness of individuals, including: (1) Incident Management; (2) Anonymous Reporting; (3) Emergency Alerts; (4) Data Collection; (5) Communication Management; (6) Event Management; (7) Health and Wellness Management; and (8) Guest and Visitor Management. These modules can have overlapping features or functions to create built-in redundancies for increased security. The cloud-based platform 150 and/or the modules are configured to connect or communicate via a network (e.g., via a communal or direct connection point, link, or path, all of which may be wired or wireless, or via other communications technologies, or a combination thereof). The network can include the Internet, a local area network (LAN), a wide area network (WAN), a virtual private network (VPN), or any other public or private means of electronic computer communication capable of transferring data between the modules, user devices, databases, or the like. The modules may include an operations management system 200, a communications system 300, a visitor management system 400, a safety operation center system 500, a safety alert system 600, a health and wellness system 700, an event management system 800, a tardy management system 900, a classroom observation management system 1000, and a survey system 1100. As discussed further below, the modules may act in conjunction with each other and may include overlapping functions for redundancy so as to increase safety, health, and wellness of all individuals.

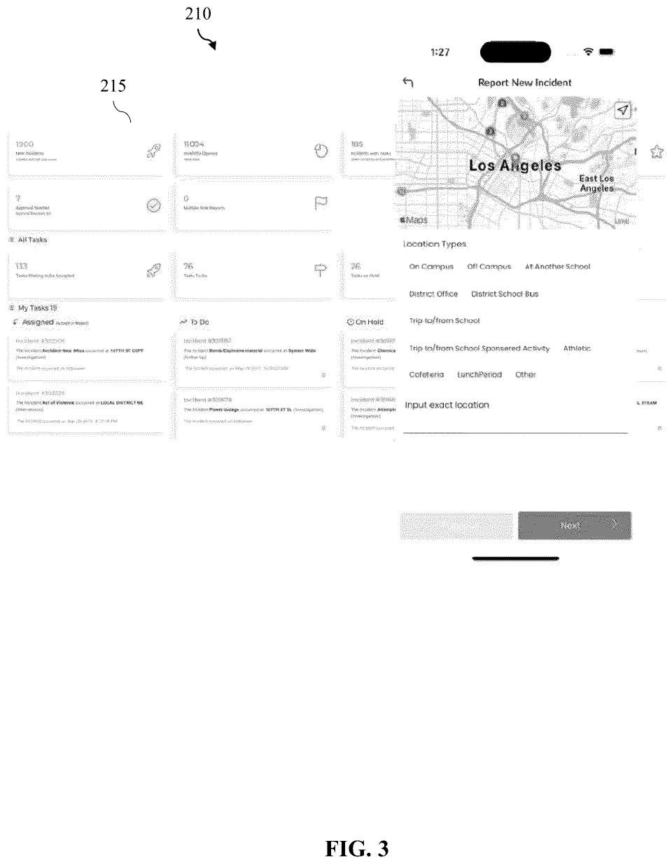

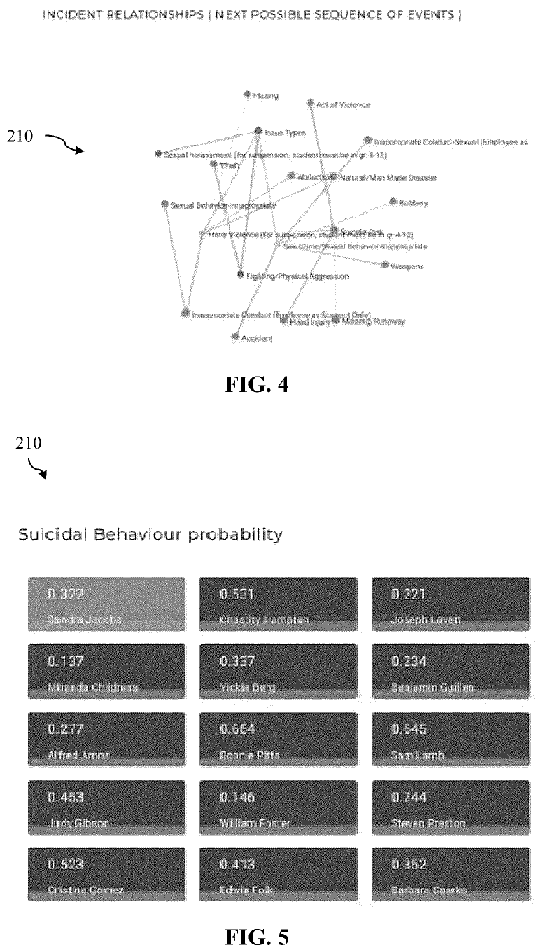

In an embodiment, the operations management system 200 includes an incident management system 210, a patrolling management system 220, and a facility and asset management system 230. As shown in FIGS. 1 and 3, the incident management system 210 is configured to collect and track student incidents (e.g., bullying, fighting, cyber incidents, etc.). These incidents may be used to predict student behavior and allow staff to engage in proactive measures to prevent future incidents, including violent incidents. As illustrated generally in FIGS. 4 and 5, the incident management system 210 may be configured to predict future events or incidents using AI models, wherein the AI model determines a possible or likely next sequence of events based on the collected student incident events and personal data. The incident management system 210 may further be configured to predict specific behavior, including suicide or self-harm, as well as behavior indicating other health conditions (e.g., eating disorders, behavioral disorders, etc.).

The incident management system 210 is further configured to monitor common school tasks (e.g., maintenance issues, software updates, etc.), incidents related to Title IX, and compliance reporting for state, federal, and/or other regulatory bodies. The incident management system 210 includes centralized dashboard 215 for performing these functions (e.g., collecting, tracking, managing, and responding to events), which allows for reduced issue resolution time and helps ensure compliance through robust back-and-forth reporting. CASES™ is an example of a case management feature for the incident management system 210.

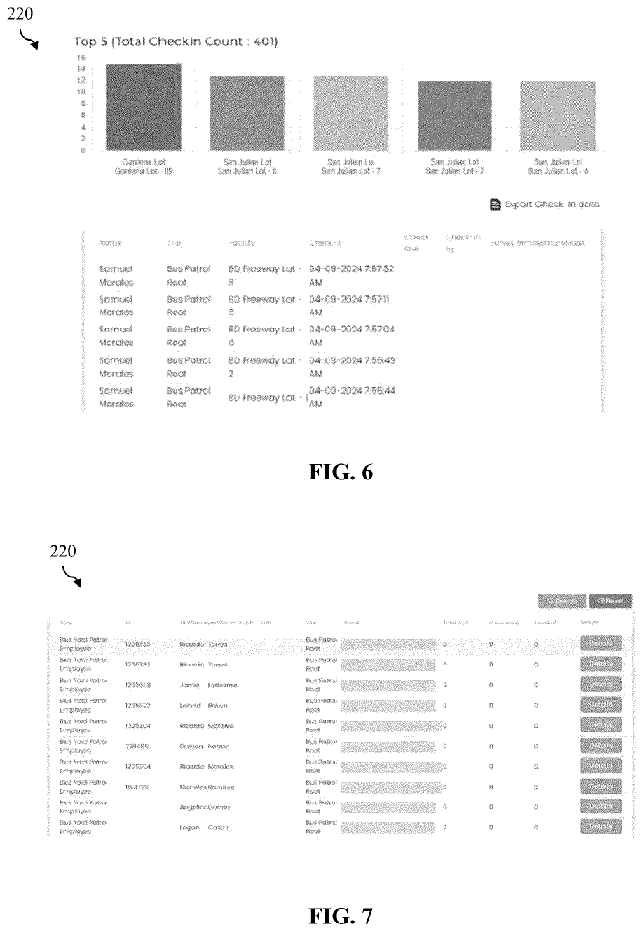

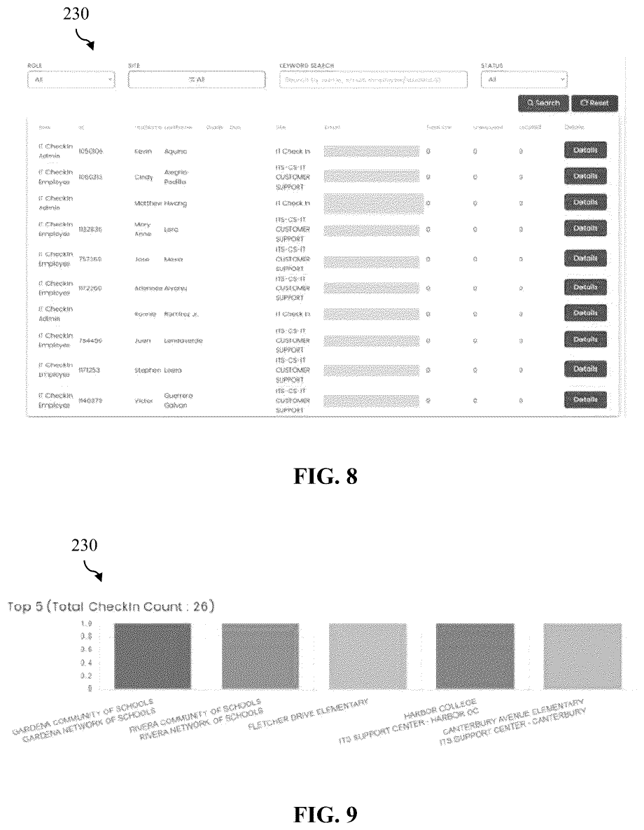

Referring to FIGS. 6 and 7, the patrolling management system 220 is configured to track and manage staff patrols or inspections of sites and facilities such that assets are being properly protected. The facility and asset management system 230 is configured to manage and monitor all facilities and assets (e.g., equipment, etc.). For example, the facility and asset management system 230 allows for the centralized and efficient management of school spaces, space reservations, and IT assets (e.g., IT equipment and software licenses). As illustrated in FIGS. 8 and 9, the facility and asset management system 230 includes applications having user-interfaces for monitoring equipment and facilities, such as a dashboard for assisting IT dispatch service all facilities of the community. ACCESS™ is an example of the patrolling management system 220 and the facility and asset management system 230.

Referring to FIG. 1, the communications system 300 includes a collection of devices, systems, processes configured to help students, staff, and parents to stay informed of important emergency and non-emergency updates. The communications system 300 includes mass communication systems 310, emergency notifications 320, and broadcasting system 330 and configured for accessibility 340 via a numerous means (e.g., mobile devices, smart devices, computing devices, or the like). Mass communication systems 310 includes communication of events, incidents, status updates, or instructions to all or select persons within and outside of the school via direct messaging (SMS, email, etc.), voice, or the like. For example, mass communication 310 can be used to inform parents of certain school events or health present concerns (e.g., contagious illness, contaminated food, etc.). Mass communication systems 310 can further be used to alert students, teachers, and other staff that an unauthorized intruder has entered the school's premise and to provide instructions for how to proceed safely. Emergency notifications 320 can be accomplished by mass communication 310 features or other means and are used to alert persons (e.g., students, staff, parents, emergency responders, etc.) of an ongoing or predicted emergency. Emergencies can include dangerous weather, armed intruders, or the like. Broadcasting systems 330 includes streaming audio or video information to a publically or privately accessible server, such as school programs, school events, camera feeds, or the like. For example, a slide show of important reminders or events for a particular month may be broadcast to a sever or platform whereby parents can quickly become informed.

In general, the communication system 300 updates parents on school events, bus delays and status updates, parent teacher conferences, fundraising events, school updates, school closures, emergencies, or the like. Similarly, students may be informed of events, school closure, meetings, emergencies, etc. The communication system 300 may also be used to keep school administrators and staff up to date on upcoming required tasks for upcoming meetings or events, routine or unexpected maintenance requests, emergencies, etc. Overall, the communication system 300 enables a school district to efficiently automate messaging tasks or mass communication to its community.

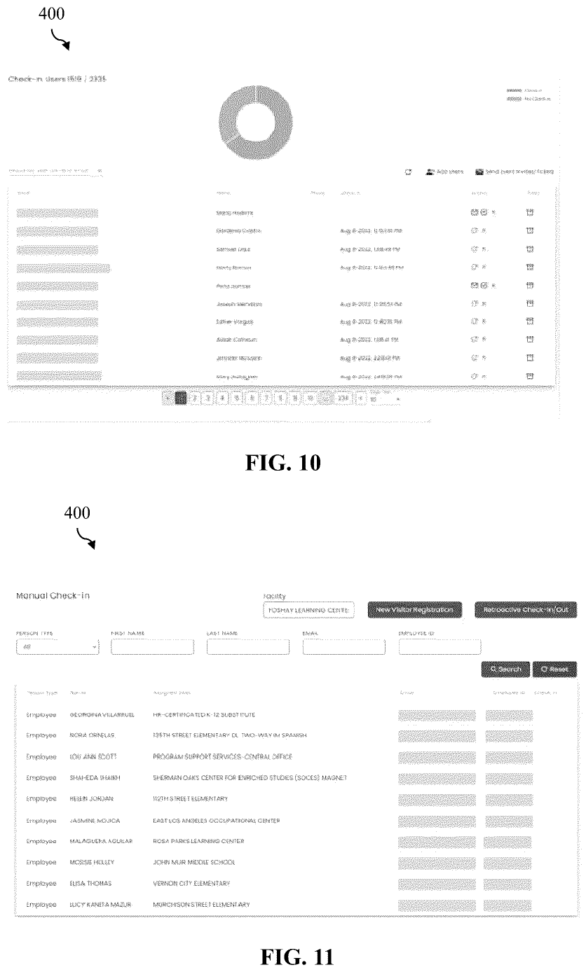

Referring to FIGS. 1, 2, and 10-12, in an embodiment, the visitor management system 400 includes a collection of devices, systems, processes configured to ensure all guests (including volunteers) have been screened and vetted, and to allow a user to track all guest locations across multiple campuses (e.g., HOST™). The visitor management system 400 includes perform guest registration 410, background check 420, and volunteer management 430. Guest registration 410 includes check-in and check-out processes. For example, a visitor will check-in via a self-service interface (e.g., a kiosk having touch screen or other computing device) and receive a badge (e.g., a digital badge). The badge can be generated by the self-service interface (e.g., the self-service interface can print a label that the visitor attaches to their person) or provided by a staff member at the check-in site. The badge may allow the guest to access the facilities (e.g., door keycard readers, etc.) and can include detailed information for allowing other staff members to visually gather key information about the guest and determine if the guest authorized to be in certain locations or with students.

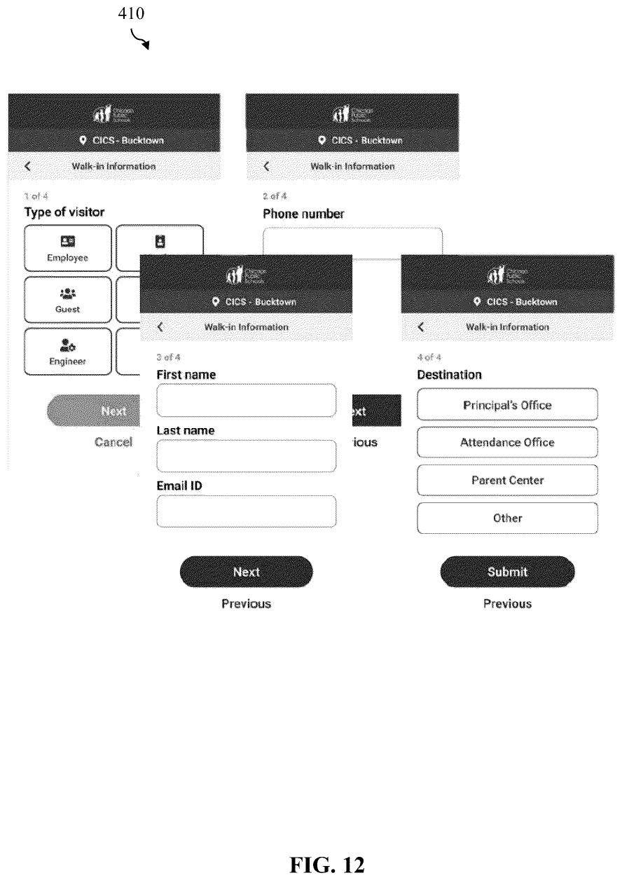

To check-in, the visitor will complete a series of questions to determine their intentions, background information, and relation to the school. For example, as shown in FIG. 12, the visitor must indicate their role, provide their contact information, provide their name, and indicate where they would like to go within the school's premises. A school district can customize the screening questionnaire depending upon their needs, time of visitation, intended location, relation, etc. In some embodiments, the guest registration 410 includes a pre-registration process where the visitor may complete the required check-in information in advance.

After the pre-registration and/or the check-in processes, the background check 420 on the visitor is performed. The background check 420 can include searching databases such as the national criminal databases like the National Crime Information Center (NCIC), state criminal databases, and the Interstate Identification Index (III), or the like. The visitor is not provided a badge unless they pass the necessary criteria on the background check 420. Similarly, volunteer management 430 includes processes like guest registration 410 and background checks 420 to ensure volunteers are safe to be around the student and to monitor where the volunteers should be located on the school's premises.

Accordingly, the visitor management system 400 reduces risk by determining if a guest should be allowed onto the premise, deterring the guest from illegal or unwanted behavior as a result of their collected personal information, and allowing for easier management of volunteers that may not commonly visit school grounds. Moreover, the visitor management system 400 enables users of the system 100 to gain situational awareness regarding guests as a result of centralized reporting and emergency management.

Referring to FIG. 1, in an embodiment, the safety operation center system 500 includes a collection of devices, systems, processes configured to provide a situation awareness system 510 including generation of heat maps identifying areas currently or historically experiencing un-safe conditions, and a sensor system 520 including camera feeds of the interior and exterior of a building, weapon detection sensors (e.g., metal detectors for detecting guns, knives, etc.), and hazard sensors (e.g., smoke alarms, etc.). The safety operation center system 500 may further include an emergency operation center 530, a cyber-incident management system 540, a ridership management system 550, and weather monitoring system 560.

The emergency operation center 530 may have access to all other modules so as to have access to all necessary information and communication systems. For example, personnel in the emergency operation center 530 can receive the situation awareness system 510, the sensor system 520, guest registration 410, background check 420, and the incident management system 210 such that the personnel can then determine whether to issue alerts via mass communication 310 or emergency notifications 320 (e.g., that a dangerous person or situation is occurring on campus), provide instructions to the patrolling management system 220 (e.g., to check a specific location that has not be routinely monitored or to more actively search for certain activities or individuals), and/or provide instructions to the facility and asset management system 230 (e.g., to section off a dangerous area that needs repair).

The cyber-incident management system 540 is configured to monitor use of school networks, cyber threats, cyber bullying, and the like. The ridership management system 550 is configured to monitor student usage of school or public vehicles (e.g., buses, or other transportation means) to ensure that students safely board and de-board from the vehicles. For example, the ridership management system 550 may include a system for registering when a student has boarded and de-boarded the vehicle, such as scanning a badge or input from an operator of the vehicle who is actively tracking students expected to use the provided transportation. The weather monitoring system 560 is configured to monitor weather and to provide alerts if certain weathers conditions are expected to occur that would result in unsafe conditions for students (e.g., icy roads causing hazardous conditions for the vehicles of the ridership management system 550, heavy rain or wind that would prevent outdoor events from being hosted, or extreme weather (hot, cold, humid, UV index, etc.) that would present a health concern for students athletes, etc.). The weather monitoring system 560 can receive weather reports from local sources outside of the school district and can be set to issues the alerts based upon a set criteria. In some embodiments, the weather monitoring system 560 issues alerts via the mass communication systems 310, emergency notifications 320, or the broadcasting system 330.

In some embodiments, the safety operation center system 500 communicates with law enforcement (e.g., state, federal, or private) and to quickly and efficiently inform law enforcement of an ongoing situation (e.g., an emergency or increase in illegal activity on or near school grounds, etc.). The safety operation center system 500 may further provide access to some or all of the modules and features of the system 100, like the situation awareness system 510, sensor system 520, the incident management system 210, or the like, such that law enforcement officers can act in the best way to resolve a threat.

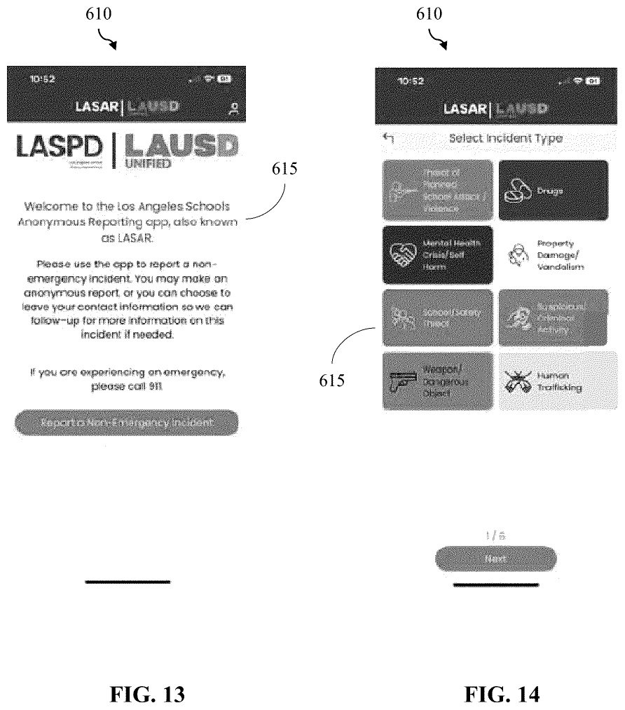

Referring to FIGS. 1 and 13-16, in an embodiment, the safety alert system 600 includes a collection of devices, systems, processes configured to allow individuals (e.g., students, teachers, administrators, parents, etc.) to report suspicious activity, threatening events, or other emergencies (e.g., medical emergencies). The safety alert system 600 includes a reporting system 610 configured to allow individuals to anonymously report suspicious or dangerous behavior (e.g., ESCALATE™). The reporting system 610 includes an application 615, text-based system, and/or web-based system for providing convent and ease-to-use means of reporting. As illustrated in FIGS. 12 and 13, the individual can use the application 615 on their mobile device or personal computer to report an incident or dangerous behavior. The individual is prompted to select an incident from a pre-selected list and is then further prompted to provide additional details (including their contact information should they so choose). The text-based system and web-based system may be used in a similar manner. By facilitating convenient and anonymous reporting, the district can stay aware of student issues that likely would not otherwise get reported and proactively prevent emergencies before they occur. In some embodiments, reporting system 610 communicates with the incident management system 210 and sends information regarding student incidents.

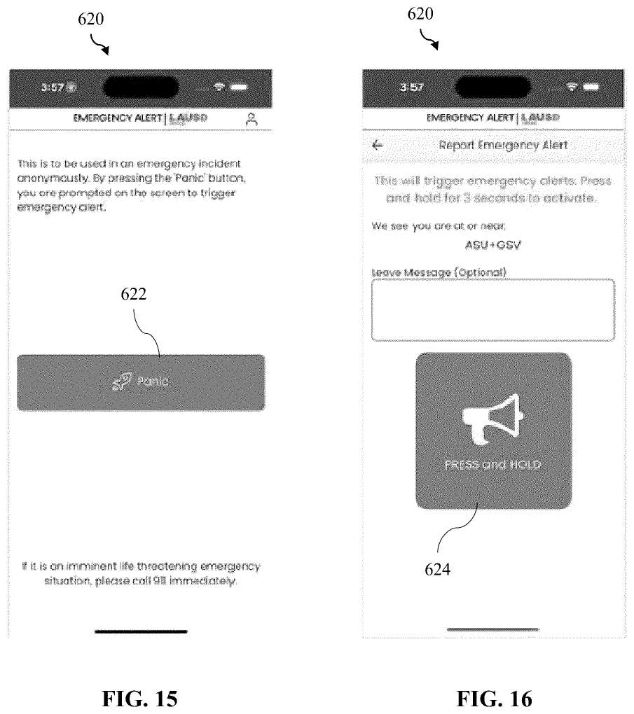

As illustrated in FIGS. 15 and 16, The safety alert system 600 further includes an emergency alert system 620, wherein an individual can anonymously use an application to issue an emergency alert by pressing and holding a “panic” button 622 (e.g., NOTIFY™). To emergency alerts are not accidently triggered, the individual may be required to press another button 624 before being presented with the panic button 622, which must be held for a set amount of time (e.g., three seconds) before the emergency alert is reported. In some embodiments, an “all clear” button, or the like, may also be included to update the status of the emergency. The emergency alert system 620 is configured to receive and/or determine a relative location of the individual at the time the emergency alert is triggered so that responders of the safety operation center system 500, law enforcement, or other persons (e.g., administrators, teachers, etc.) are aware of where the emergency is occurring despite being anonymously reported. In addition, the individual can leave a message outlining details of the emergency. The individual is also promoted to call 911 (i.e., local law enforcement or emergency services) for imminent life threatening emergency situations.

Consequently, the emergency alert system 620 provides a two-way communication method of reporting an emergency and helps ensure the safety of all community members (e.g., all students and staff members) by enabling them to call for help during times of an extreme emergency. Furthermore, the safety alert system 600 provides an alternative method for the district to promptly and efficiently react to emergencies, in addition to the functions of the safety operation center system 500.

As discussed above, the operations management system 200, the communications system 300, the visitor management system 400, the safety operation center system 500, and the safety alert system 600 act collectively to ensure the safety and well-being of all community members. Any overlapping functions of these modules adds redundancy to the system 100 so as to ensure that critical information is not lost, ignored, or mishandled.

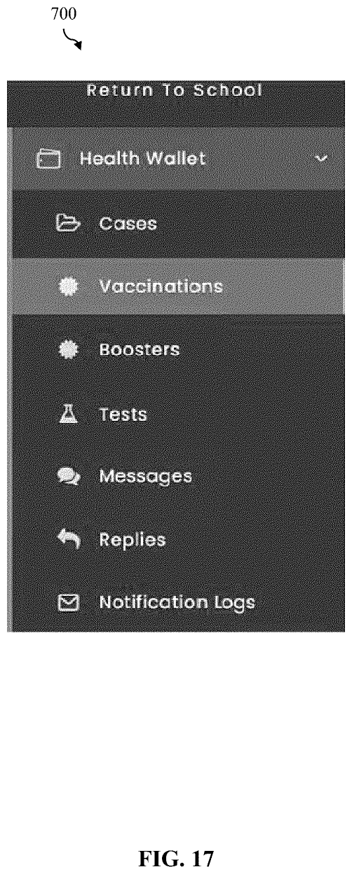

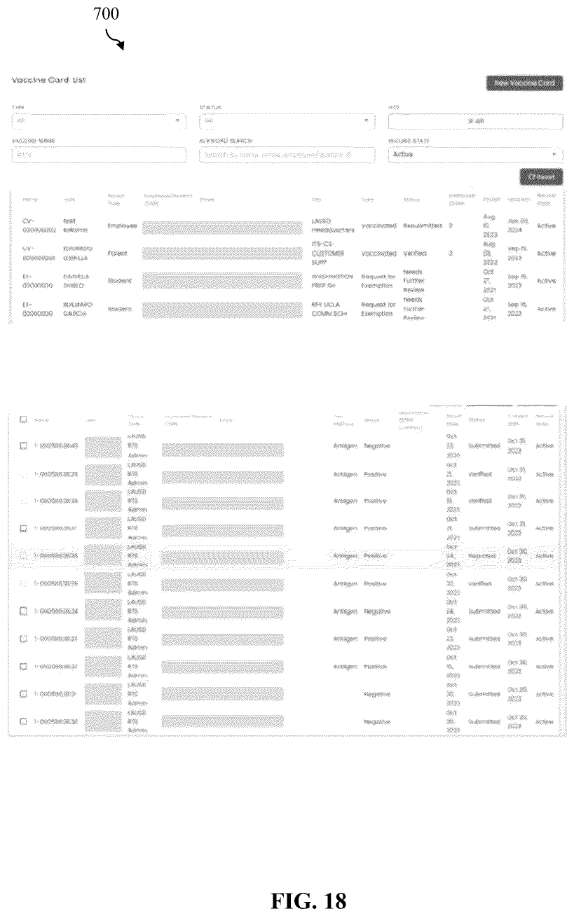

As illustrated in FIGS. 17 and 18, the health and wellness system 700 includes a collection of staff, devices, systems, processes configured to securely manage student health and wellbeing including mental health screenings and records management (e.g., vaccination records), in compliance with HIPAA requirements (e.g., WELLNES™). For example, immunization records can be digitalized for compliance and security, and the use a centralized dashboard allows for easy tracking, robust reporting, reminders, and workflow efficiencies across multiple sites. The health and wellness system 700 includes intuitive and capable software that is hierarchy aware, multi-lingual, API-enabled and features single sign on. The health and wellness system 700 also includes telehealth services 710 for providing increased access to health services for community members (TELEHEALTH™). In an embodiment, the health and wellness system 700 includes a predictive AI model for determining the physical and mental health of students.

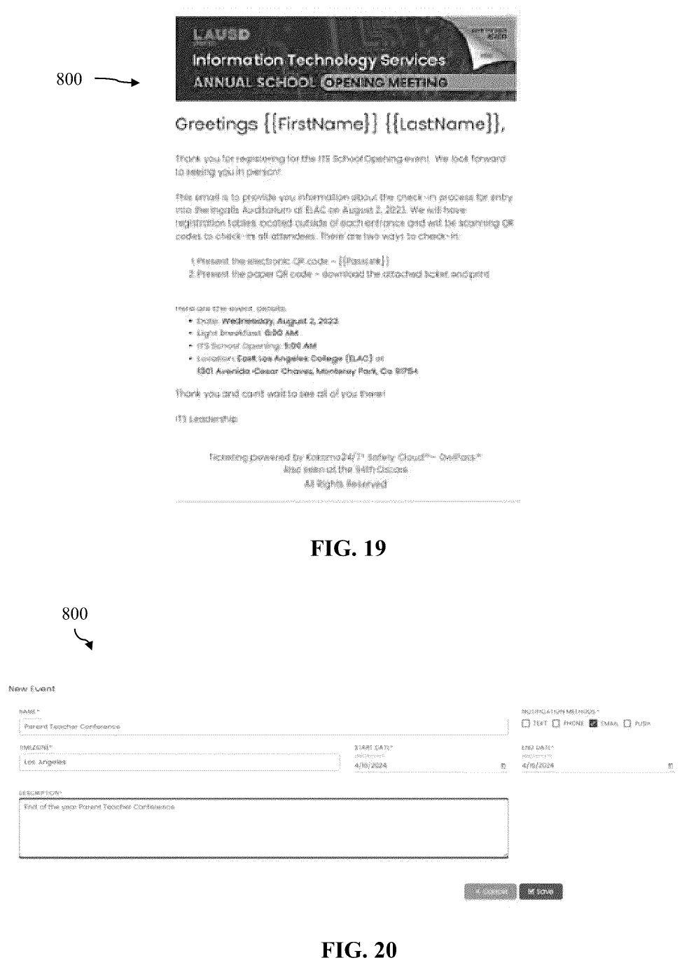

As illustrated in FIGS. 19 and 20, the event management system 800 allows for a single comprehensive means of planning and managing meetings, conferences and live events (e.g., OWLPASS™). The event management system 800 is configured to utilize digital credentials to manage clearance into events using a QR code, collect pre-screening information for digital badging, and create custom forms and notifications for attendees. The event management system 800 is configured to individually, or in conjunction with the visitor management system 400, to collect pre-screening information for the issuance of digital badges to guests of the event. In this way, the event management system 800 is also operable to perform diagnostic testing on the guests and event conditions to increase safety of attendees. The event management system 800 may include an emergency alert button for critical situations that can be triggered by staff in the event of an emergency, wherein local emergency services will be immediately contacted and notified of the emergency. The event management system 800 includes the issuance and management of tickets.

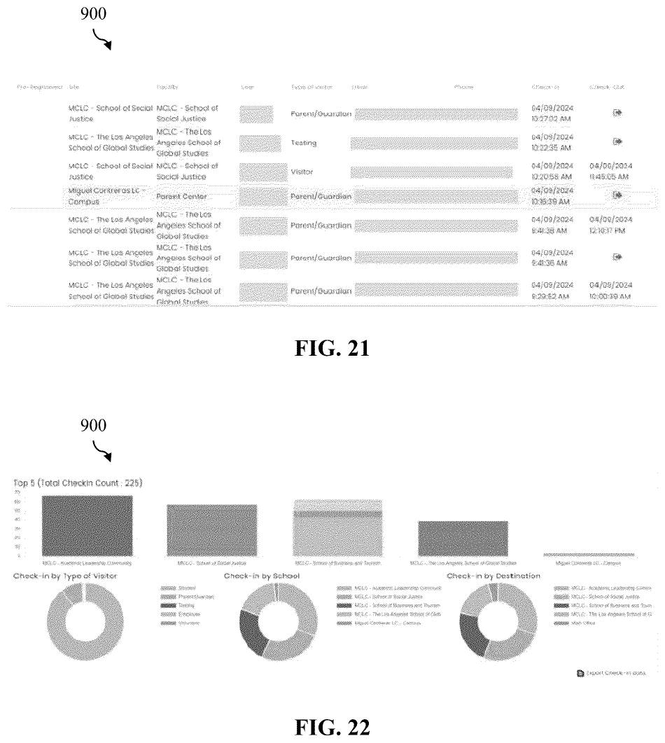

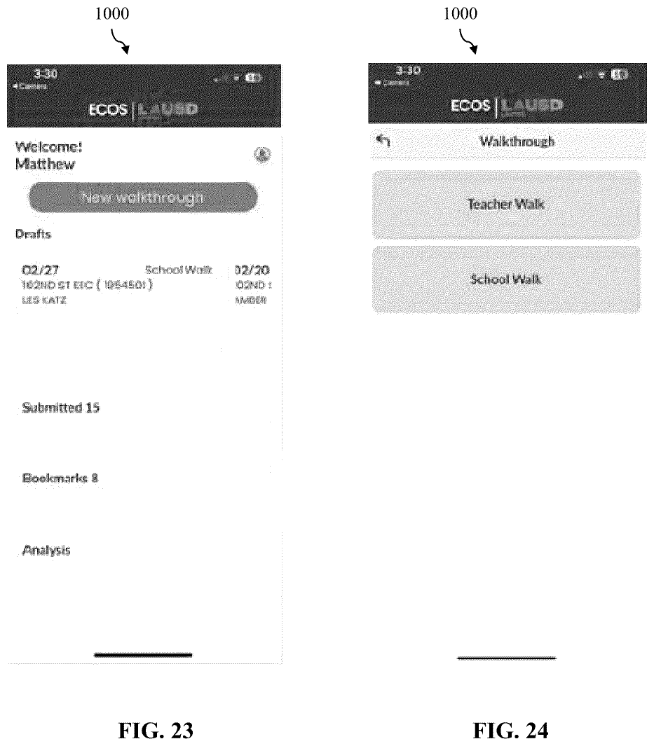

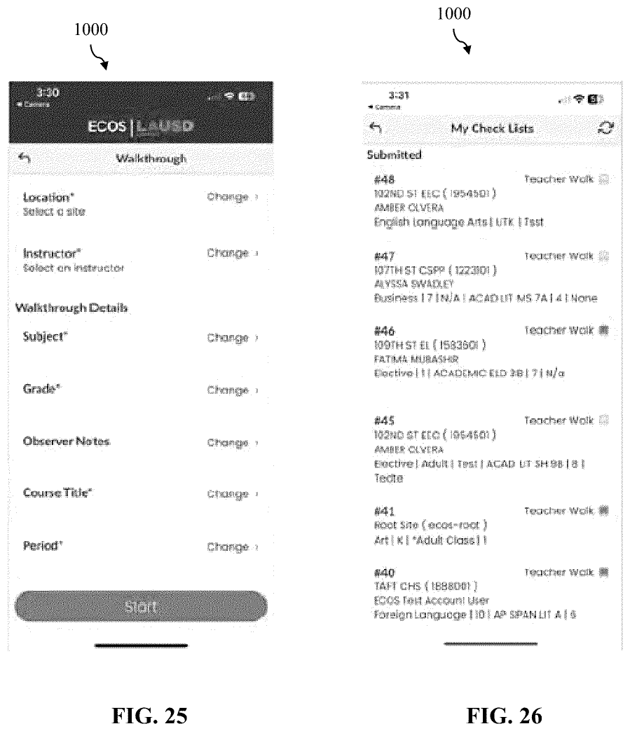

Referring to FIGS. 21 and 22, in an embodiment, the tardy management system 900 is configured to monitor the attendance of students and to determine if a student is on school grounds so that they may be accounted for in the event of an emergency. Referring to FIGS. 23-26, in an embodiment, the classroom observation management system 1000 is configured to collect and monitor all data points involving instructional practices of teachers, the scholastic performance of a school, etc. For example, the classroom observation management system 1000 includes rubrics for conducting walkthroughs and is configured to generate a report and determine appropriate feedback based on data points collected from the walkthrough.

In addition to the modules discussed above, data collection can also be accomplished by the survey system 1100. In an embodiment, the survey system 1100 includes customizable surveys, wherein the information received from the surveys is communicated to the other modules. For example, the survey system 1100 includes surveys for mental health and well-being checks, collecting student and teacher feedback, and threat assessment.

Referring to FIGS. 2, in and embodiment a method 2000 can be performed by the above system 100 and cloud-based platform 150. The method 2000 includes managing an event or access to a facility via the event management system 800, wherein tickets are issued for entry into the event, at step 2010. At 2020 checking-in of visitors includes the visitor management system 400, wherein the visitors check-in via the self-service kiosk. At 2025, if the visitor has a ticket and is cleared by the visitor management system 400, the visitor receives a digital badge. At 2030, if the visitor enters the facility without a ticket or without being cleared by the visitor management system 400, the communication system 300 informs students and staff of the unauthorized visitor via the mass communication systems 310, emergency notifications 320, and/or the broadcasting system 330. At 2040 the students and staff are evacuated from the facility according to a predetermined evacuation plan and the location of the unauthorized visitor or are instructed to perform another action (e.g., run, hide, or fight). The communication system 300 and/or the safety operation center system 500 notifies law enforcement of the emergency. At 2050, after the emergency has concluded, students and staff are provided with access to the health and wellness system 700 for managing student and staff health and wellbeing after a potentially traumatic event. At 2060, the safety operation center system 500 monitors the facility, authorized visitors, and unauthorized visitors. At 2070, an authorized visitor checks-out by scanning their digital badge at the self-service kiosk.

The systems and methods described herein may be configured to provide centralized management, data collection, and emergency response for a school, business, or the like.

Clause 1. A cloud-based system for facilitating health, safety, and wellness of individuals in a user community. The system may include an incident management system having a centralized dashboard for collecting, tracking, managing, and responding to events; an anonymous reporting system for providing individual users in the user community with a safe and private means for reporting concerning behavior; an emergency alert system including a mobile application with an emergency alert button for indicating there is an emergency; a data collection system including customizable surveys to collect information from community users; a communication management system to keep community users informed; and a visitor management system including a guest registration system and a background check for ensuring a visitor is safe through screening and tracking.

Clause 2. The cloud-based system as clause 1 describes where the community is one of either a school, a business, a facility, a venue, or a campus.

Clause 3. The cloud-based system as either of clauses 1 or 2 describe where the incident management system is configured to predict future incidents using artificial intelligence (AI), where the AI is programmed to determine a possible or likely next sequence of events based on collected incident events and personal data.

Clause 4. The cloud-based system as any of clauses 1-3 describe further may include a patrolling management system configured to track and manage patrols or inspections of sites and facilities to ensure community assets are protected, and a facility and asset management system configured to manage and monitor facilities and assets.

Clause 5. The cloud-based system as any of clauses 1-4 describe where the communication management system includes a mass communication system configured to communicate events, incidents, status updates, or instructions to selected persons within and outside of a school via direct messaging, voice communication, or similar means; where the mass communication system is operable to inform parents of school events or health concerns and to alert students, teachers, and staff of unauthorized intruders; an emergency notification system configured to alert persons of ongoing or predicted emergencies, where the emergency notification system utilizes features of the mass communication system or other communication means to notify students, staff, parents, and emergency responders of emergencies including dangerous weather or armed intruders; and a broadcasting system configured to stream audio or video information to a publicly or privately accessible server, where the broadcasting system is operable to broadcast school programs, events, and camera feeds and to display important reminders or events to parents.

Clause 6. The cloud-based system as any of clauses 1-5 describe where the communication management system is configured to update users about school events, bus delays, school closures, and emergencies.

Clause 7. The cloud-based system as any of clauses 1-6 describe where the guest registration system includes a self-service interface for the visitor to provide their relation to or role within the community, contact information, and intended location, and where the self-service interface is configured to provide the visitor with a badge for accessing the facilities.

Clause 8. The cloud-based system as any of clauses 1-7 describe further may include a safety operation center system for monitoring the community and communicating with law enforcement, the safety operation center system including a sensor system having cameras and weapon detectors, and a cyber-incident management system configured to monitor the use of community networks, cyber threats, and cyberbullying.

Clause 9. The cloud-based system as any of clauses 1-8 describe where the safety operation center system includes a ridership management system configured to monitor user usage of community vehicles to ensure that a user safely boards and de-boards from the vehicles, and a situation awareness system configured to generate heat maps identifying areas currently or historically experiencing unsafe conditions.

Clause 10. A method for facilitating health, safety, and wellness of individuals in a user community. The method may include the steps of managing incidents using a centralized dashboard for collecting, tracking, managing, and responding to events; allowing anonymous reporting by providing individual users in the user community; providing an emergency alert system including a digital emergency alert button for indicating there is an emergency; collecting data from community users by customizable surveys; managing communication to keep community users informed with important update information; and managing visitors to ensure all visitors are safe and monitored while at the community through screening and tracking.

Clause 11. The method as clause 10 describes where the community is one of either a school, a business, a facility, a venue, or a campus.

Clause 12. The method as either of clauses 10 or 11 describe where managing incidents includes predicting future incidents using artificial intelligence (AI), where the AI is programmed to determine a possible or likely next sequence of events based on collected incident events and personal data.

Clause 13. The method as any of clauses 10-12 describe further may include managing patrolling of inspections of sites and facilities to ensure community assets are protected using the centralized dashboard, and managing facilities and assets using the centralized dashboard.

Clause 14. The method as any of clauses 10-13 describe where managing communication includes mass communication with the community, providing emergency notifications, and providing a broadcast system.

Clause 15. The method as any of clauses 10-14 describe further may include managing users' usage of community vehicles for ensuring that users safely board and de-board the vehicles, and generating heat maps for identifying areas currently or historically experiencing unsafe conditions.

Clause 16. A cloud-based system for facilitating health, safety, and wellness of individuals in a user community. The system may include an incident management system having a centralized dashboard for collecting, tracking, managing, and responding to events, where the incident management system is configured to predict future incidents using artificial intelligence (AI) and where the AI is programmed to determine a possible or likely next sequence of events based on collected incident events and personal data; a safety alert system including an anonymous reporting system for allowing users to anonymously report incidents and an emergency alert system for remotely indicating a user is experiencing an emergency; a safety operation center system for monitoring the community and communicating with law enforcement, where the safety operation center system includes a cyber-incident management system configured to monitor the use of community networks, cyber threats, and cyberbullying; and a ridership management system configured to monitor user usage of community vehicles to ensure that a user safely boards and de-boards from the vehicles.

Clause 17. The cloud-based system as clause 16 describes where the safety operation center system includes a situation awareness system configured to generate heat maps identifying areas currently or historically experiencing unsafe conditions.

Clause 18. The cloud-based system as either of clauses 16 or 17 describe further may include a health and wellness system configured to securely manage student health and wellbeing.

Clause 19. The cloud-based system as any of clauses 16-18 describe where the health and wellness system includes telehealth services.

Clause 20. The cloud-based system as any of clauses 16-19 describe further may include a classroom observation management system configured to collect and monitor all data points involving performance metrics of staff and of a school.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

The word “example” is used herein to mean serving as an example, instance, or illustration. Any aspect or design described herein as “example” is not necessarily to be construed as preferred or advantageous over other aspects or designs. Rather, use of the word “example” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an implementation” or “one implementation” throughout is not intended to mean the same embodiment or implementation unless described as such.

Implementations of the systems, algorithms, methods, instructions, etc., described herein can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

In embodiments, a processor may include processor hardware (shared, dedicated, or group) that executes code and memory hardware (shared, dedicated, or group) that stores code executed by the processor hardware. The code is configured to provide the features of the methods and systems described herein. To carry out the control commands provided herein, a controller may include the processor 1, memory, and non-volatile storage. The processor may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on computer-executable instructions residing in memory. The memory may include a single memory device or a plurality of memory devices including, but not limited to, random access memory (“RAM”), volatile memory, non-volatile memory, static random-access memory (“SRAM”), dynamic random-access memory (“DRAM”), flash memory, cache memory, or any other device capable of storing information. The non-volatile storage may include one or more persistent data storage devices such as a hard drive, optical drive, tape drive, non-volatile solid-state device, or any other device capable of persistently storing information. The processor may be configured to read into memory and execute computer-executable instructions embodying one or more software programs residing in the non-volatile storage. Programs residing in the non-volatile storage may include or be part of an operating system or an application, and may be compiled or interpreted from computer programs created using a variety of programming languages and/or technologies, including, without limitation, and either alone or in combination, Java, C, C++, C#, Objective C, Fortran, Pascal, Java Script, Python, Perl, and PL/SQL. The computer-executable instructions of the programs may be configured, upon execution by the processor 106, to output control signals to control the emitter to selectively excite the coating at a location to a more opaque state, for example.

Implementations of the subject matter and the operations described in this specification can be implemented in digital electronic circuitry, or in computer software embodied on a tangible medium, firmware, or hardware, including the structures disclosed in this specification and their structural equivalents, or in combinations of one or more of them. Implementations of the subject matter described in this specification can be implemented as one or more computer programs embodied on a tangible medium, e.g., one or more modules of computer program instructions, encoded on one or more computer storage media for execution by, or to control the operation of, a data processing apparatus. A computer storage medium can be, or be included in, a computer-readable storage device, a computer-readable storage substrate, a random or serial access memory array or device, or a combination of one or more of them. The computer storage medium can also be, or be included in, one or more separate components or media (e.g., multiple CDs, disks, or other storage devices). The computer storage medium may be tangible and non-transitory.

As used herein, the term module can include a packaged functional hardware unit designed for use with other components, a set of instructions executable by a controller (e.g., a processor executing software or firmware), processing circuitry configured to perform a particular function, and a self-contained hardware or software component that interfaces with a larger system. For example, a module can include an application specific integrated circuit (ASIC), a Field Programmable Gate Array (FPGA), a circuit, digital logic circuit, an analog circuit, a combination of discrete circuits, gates, and other types of hardware or combination thereof. In other embodiments, a module can include memory that stores instructions executable by a controller to implement a feature of the module.

Further, in one aspect, for example, systems described herein can be implemented using a general-purpose computer or general-purpose processor with a computer program that, when executed, carries out any of the respective methods, algorithms, and/or instructions described herein. In addition, or alternatively, for example, a special purpose computer/processor can be utilized which can contain other hardware for carrying out any of the methods, algorithms, or instructions described herein.

Further, all or a portion of implementations of the present disclosure can take the form of a computer program product accessible from, for example, a computer-usable or computer-readable medium. A computer-usable or computer-readable medium can be any device that can, for example, tangibly contain, store, communicate, or transport the program for use by or in connection with any processor. The medium can be, for example, an electronic, magnetic, optical, electromagnetic, or a semiconductor device. Other suitable mediums are also available.

The above-described embodiments, implementations, and aspects have been described in order to allow easy understanding of the present invention and do not limit the present invention. On the contrary, the invention is intended to cover various modifications and equivalent arrangements included within the scope of the appended claims, which scope is to be accorded the broadest interpretation so as to encompass all such modifications and equivalent structure as is permitted under the law.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the disclosure. Additionally, while various embodiments of the disclosure have been described, it is to be understood that aspects of the disclosure may include only some of the described embodiments. Accordingly, the disclosure is not to be seen as limited by the foregoing description.

What is claimed is:

1. A cloud-based system for facilitating health, safety, and wellness of individuals in a user community, the system comprising:
an incident management system having a centralized dashboard for collecting, tracking, managing, and responding to events;
an anonymous reporting system for providing individual users in the user community with a safe and private means for reporting concerning behavior;
an emergency alter system including a mobile application with an emergency alert button for indicating there is an emergency;
a data collection system including customizable surveys to collect information from community users;
a communication management system to keep community users informed; and
a visitor management system including a guest registration system and a background check for ensuring a visitor is safe through screening and tracking.

2. The cloud-based system of claim 1 wherein the community is one of either a school, a business, a facility, a venue, or a campus.

3. The cloud-based system of claim 1 wherein the incident management system is configured to predict future incidents using artificial intelligence (AI), wherein the AI is programed to determine a possible or likely next sequence of events based on collected incident events and personal data.

4. The cloud-based system of claim 1 further comprising:
a patrolling management system configured to track and manage patrols or inspections of sites and facilities to ensure community assets are protected; and
a facility and asset management system configured to manage and monitor facilities and assets.

5. The cloud-based system of claim 1 wherein the communication management system includes:
a mass communication system configured to communicate events, incidents, status updates, or instructions to selected persons within and outside of a school via direct messaging, voice communication, or similar means, wherein the mass communication system is operable to inform parents of school events or health concerns, and to alert students, teachers, and staff of unauthorized intruders;
an emergency notification system configured to alert persons of ongoing or predicted emergencies, wherein the emergency notification system utilizes features of the mass communication system or other communication means to notify students, staff, parents, and emergency responders of emergencies including dangerous weather or armed intruders; and
a broadcasting system configured to stream audio or video information to a publicly or privately accessible server, wherein the broadcasting system is operable to broadcast school programs, events, and camera feeds, and to display important reminders or events to parents.

6. The cloud-based system of claim 5 wherein the communication management system is configured to update users about school events, bus delays, school closures, and emergencies.

7. The cloud-based system of claim 1 wherein the guest registration system includes a self-service interface for the visitor to provide their relation to or role within the community, contact information, and intended location; and
wherein the self-service interface is configured to provide the visitor with a badge for accessing the facilities.

8. The cloud-based system of claim 1 further comprising:
a safety operation center system for monitoring the community and communicating with law enforcement, the safety operation center system including:
a sensor system having cameras and weapon detectors; and
a cyber-incident management system configured to monitor the use of community networks, cyber threats, and cyber bullying.

9. The cloud-based system of claim 8 wherein the safety operation center system includes:
a ridership management system configured to monitor user usage of community vehicles to ensure that a user safely boards and de-boards from the vehicles; and
a situation awareness system configured to generate heat maps identifying areas currently or historically experiencing unsafe conditions.

10. A method for facilitating health, safety, and wellness of individuals in a user community, the method comprising the steps of:
managing incidents using a centralized dashboard for collecting, tracking, managing, and responding to events;
allowing anonymous reporting by providing individual users in the user community;
providing emergency alter system including a digital emergency alert button for indicating there is an emergency;
collecting data from community users by customizable surveys;
managing communication to keep community users informed with important update information; and
managing visitors to ensure all visitors are safe and monitored while at the community through screening and tracking.

11. The method of claim 10 wherein the community is one of either a school, a business, a facility, a venue, or a campus.

12. The method of claim 10 wherein managing incidents includes predicting future incidents using artificial intelligence (AI), wherein the AI is programed to determine a possible or likely next sequence of events based on collected incident events and personal data.

13. The method of claim 10 further comprising:
managing patrolling of inspections of sites and facilities to ensure community assets are protected using the centralized dashboard; and
managing facilities and assets using the centralized dashboard.

14. The method of claim 10 wherein managing communication includes mass communication with the community, providing emergency notifications, and providing a broadcast system.

15. The method of claim 10 further comprises:
managing users usage of community vehicles for ensuring that users safely boards and de-boards the vehicles; and
generating heat maps for identifying areas currently or historically experiencing unsafe conditions.

16. A cloud-based system for facilitating health, safety, and wellness of individuals in a user community, the system comprising:
an incident management system having a centralized dashboard for collecting, tracking, managing, and responding to events, wherein the incident management system is configured to predict future incidents using artificial intelligence (AI), and wherein the AI is programed to determine a possible or likely next sequence of events based on collected incident events and personal data;
a safety alert system including an anonymous reporting system for allowing users to anonymously report incidents and an emergency alter system for remotely indicating a user is experiencing an emergency;
a safety operation center system for monitoring the community and communicating with law enforcement, wherein the safety operation center system includes:
a cyber-incident management system configured to monitor the use of community networks, cyber threats, and cyber bullying; and
a ridership management system configured to monitor user usage of community vehicles to ensure that a user safely boards and de-boards from the vehicles.

17. The cloud-based system of claim 16 wherein the safety operation center system includes a situation awareness system configured to generate heat maps identifying areas currently or historically experiencing unsafe conditions.

18. The cloud-based system of claim 16 further comprising a health and wellness system configured to securely manage student health and wellbeing.

19. The cloud-based system of claim 18 wherein the health and wellness system includes telehealth services.

20. The cloud-based system of claim 18 furthering comprising a classroom observation management system configured to collect and monitor all data points involving performance metrics of staff and of a school.
